(19) United States
(12) Patent Application Publication
(10) Pub. No.: US 2025/0302672 A1
(43) Pub. Date: Oct. 2, 2025

(54) REUSABLE SANITARY AND INCONTINENCE ARTICLE

(71) Applicant: SEAMPRINT GMBH & CO. KG, Münsing (DE)

(72) Inventors: Udo SIEBERT, Münsing (DE); Inge SIEBERT, Münsing (DE)

(21) Appl. No.: 19/110,876

(22) PCT Filed: Jun. 5, 2023
(86) PCT No.: PCT/EP2023/065016

§ 371 Date: Mar. 12, 2025

(30) Foreign Application Priority Data
Sep. 13, 2022 (WO) PCT/EP2022/075381

(51) Int. Cl. A61F 13/15; A61F 13/475; A61F 13/476; A61F 13/49; A61F 13/494; A61F 13/505

(52) U.S. Cl. CPC A61F 13/15268; A61F 13/4755; A61F 13/476; A61F 13/49006; A61F 13/49017; A61F 13/49406; A61F 13/505; A61F 2013/15276

(57) ABSTRACT

The present invention relates primarily to a reusable sanitary or incontinence article having a multilayer structure comprising an inner layer including a gusset area nearest to a wearer's skin for taking up body fluid from the wearer and/or transporting the body fluid away from the wearer; an outer layer including a sealing portion for providing a barrier for the body fluid and side edge portions; an absorbent core between the inner layer and the outer layer; and a fluid tight joint connecting the inner layer, the outer layer and optionally the absorbent core to each other along at least a part of a perimeter of the gusset area; wherein the sealing portion is composed of a textile coated with an adhering coating material that forms a contiguous, impervious coating extending at least over the gusset area into the side edge portions, and wherein the side edge portions extend from opposite sides outside the gusset area, are folded over side edges of and connected to the inner layer forming a fluid tight joint between the inner layer and the outer layer along side edges of the gusset area.

### TECHNICAL FIELD OF THE INVENTION

The present invention relates to reusable, washable and launderable sanitary or incontinence articles, more particularly to reusable sanitary or incontinence articles having multilayer structures including a sealing portion composed of a textile coated with an elastomer, reusable sanitary or incontinence undergarments configured to removably accept an absorbent, and corresponding production methods.

### BACKGROUND OF THE INVENTION

200 million people worldwide are affected by incontinence, an inability of the body to control the evacuative functions of urination or defecation, likely to rise with the growing number of elderly persons in the community. In 2022, the turnover for incontinence articles is expected to amount to more than 3 billion Euro in Europe alone.

Beyond incontinence as an actual disease or syndrome, articles capable of absorbing fluid including blood are regularly needed by menstruating women. Feminine hygiene products are personal care products used during menstruation, vaginal discharge, and other bodily functions related to the vulva and vagina. Products that are used during menstruation may also be called menstrual hygiene products or sanitary articles.

Most incontinence and sanitary articles are disposable. Some are reusable. Reusable articles must be possible to thoroughly wash and to dry in a reasonable amount of time. These criteria are often contradictory, and the outcome is either an article, which is easy to dry but does not absorb a large volume of liquid or a sanitary product that takes a long time to dry but has good absorption capacity. This problem may be overcome by drying in a tumble dryer. However, there are no sanitary products commercially available that can be repeatedly washed and dried at 90° C. without impairing their integrity and performance. Due to the washability 60° to 90° bacteria are killed, providing the advantage that antibacterial coatings can be dispensed with. It should be noted that bacterial coatings that are worn directly on the skin or in another layer, such as underwear or inserts is controversial for environmental and health reasons. In addition, most reusable products use a membrane as water barrier. Disadvantages of this include poor breathability, short life, low heat resistance, and a perceptible rustling noise.

Thus, an object of the present invention is to mitigate the above problems. In particular, it is an object to provide a reusable sanitary or incontinence article which can be repeatedly washed and dried efficiently, which is comfortable, maintains discreteness (i.e. makes little noise) and maintains fluid retaining performance. A further object is to keep complexity, and hence production costs, of the article at a minimum. These, and other objects which will become apparent from the following, are accomplished by means of the present invention as defined in the accompanying claims.

### SUMMARY OF THE INVENTION

In a first aspect, the present invention concerns a reusable sanitary or incontinence article having a multilayer structure. The article comprises an inner layer including a gusset area nearest to a wearer's skin for taking up body fluid from the wearer, an outer layer including a sealing portion extending at least over the gusset area and providing a barrier for the body fluid, and an absorbent core between the inner layer and the outer layer, the absorbent core including one or more absorbent layers. A fluid tight joint connects the inner layer, the outer layer and optionally the absorbent core to each other along a perimeter of the gusset area. The sealing portion is composed of a textile coated with an adhering coating material that has sealing properties such as an adhesive or sealant. A self-adhering elastomer, in particular silicone, is preferred. The elastomer used, for example, is a thermoplastic elastomer that becomes soft or liquid when heated and hardens when cooled. When heated, this elastomer can be applied in dots, lines or areas and penetrates into the fabric of the fabric layer so that, after the elastomer has cured, there is a strong and permanent bond with the fabric layer. Other coatings that can be used include rubber, polyvinyl chloride, polyurethane, fluoropolymers and/or wax. The coating is preferably contiguous at least in the gusset area. However, it can also be applied over the entire surface or consist of several partial surfaces that are sealed to each other.

In one embodiment, the outer layer includes side edge portions extending from opposite sides outside the gusset area, folded over side edges of and connected to the inner layer. This means, opposite side edge portions (left and right portions from the perspective of the wearer) of the outer layer are folded over side edges of the inner layer and connected thereto. The joint that connects the folded portions to the inner layer is preferably the same joint that connects the outer layer to the inner layer referred to above, but may also be a separate joint.

In a further embodiment, the side edge portions of the outer layer are sealingly connected to the inner layer. For instance, one or more layers of the multilayer structure may be adhered to each other using an adhering coating material, preferably elastomer, in particular silicone. In this embodiment, the side edge portions do neither need to extend from opposite sides outside the gusset area, nor need to be folded over side edges.

The present invention is based on the innovative finding that a, preferably contiguous, coating renders textile impervious to body fluids. A membrane is not required. As mentioned above, articles with membranes make a rustling noise as the person moves. In contrast, the sanitary or incontinence article of the invention makes little noise and can be repeatedly washed and dried at up to 90° C., without losing fluid retaining performance. The coating is capable of penetrating the textile structure and thus adheres thereto without any further means. The coated textile can thus be handled like a single layer. This is a great advantage over membrane-based technologies. Handling of the coated textile during production of the article of the invention is simplified. Moreover, a stretchable layer of waterproofed textile is obtained. In addition, folded and/or sealed side edge portions help to prevent that body fluid spreads outside the sealing portion and creates a wet feeling on the skin.

The inner layer takes up and rapidly transports the body fluid into the interior of the multilayer structure. The inner layer has an outer surface facing away from the absorbent core and an inner surface facing towards the absorbent core. Its surface structure is preferably perforated but can also be textured or smooth. The inner layer is preferably formed from a textile. The term “textile” as used herein denotes a woven, knitted or stitch-bonded, or any otherwise prepared fabric. The textile can be made from natural fibers, from synthetically or chemically produced threads and also includes mixed forms.

When the outer layer has an outer surface facing away from the absorbent core and an inner surface facing towards the absorbent core, only the outer surface, or only the inner surface, or both the inner and outer surfaces, of the outer layer may be coated with the adhering coating material, preferably elastomer, in particular silicone.

The main purpose of the coating is to render the outer layer impervious for body fluid. To this end, the coating is applied (preferably contiguously) over at least the entire area overlying the gusset area. Optionally, the coating extends outside the gusset area. The preferred elastomer is silicone. Silicone is available in medical grade. It has substantially no allergenic activity. Preferably, the coating is adhered to the outer layer by screen printing, but other manufacturing process, such as rolling, spraying, transfer printing, are envisaged, too. This results in a very uniform coating.

The textile itself of the outer layer provides a planar support for the elastomer coating and a common appearance. The textile can be made of natural fibers, of synthetically or chemically produced threads, and can also include mixed forms.

Typically, the gusset area can be described as substantially rectangular, in particular pyramidal or hourglass-shaped, or any other shape, with a mayor, longitudinal axis extending towards the front and the rear and a minor, axial axis extending towards the sides (towards the left and the right), relative to the perspective of a wearer, and can have a one-part or multi-part design. As already mentioned above, the sealing portion may extend outside the gusset area. Specifically, the sealing portion may extend outside the gusset area beyond opposite side edges of the gusset area, or the sealing layer may extend outside the gusset area beyond the front and the rear edges of the gusset area. Preferably, the sealing portion extends outside the gusset area beyond the side edges of the gusset area.

In a preferred embodiment of the invention, the outer layer includes flock embedded in and sticking out of the elastomer coating. Flocking is well known to the skilled person. Several advantages are associated with a flocked outer layer, in particular a flocked sealing portion. If the flock attaches to the inner surface of the sealing portion, the flock helps to absorb body fluid and thus serve as a sole or an additional means of absorption. As already mentioned above, some known types of incontinence articles make a rustling noise as the person moves, arising from plastic membranes around the absorbent core. Thus, flock attached to the inner surface of the outer layer may further help to reduce such noise. On the other hand, if the flock attaches to the outer surface of the outer layer, the flock may not only reduce crackling sounds but may also help to keep the outer layer in place, i.e. help to prevent slipping of the sanitary or incontinence article. This is particularly useful when the article is a liner or a pad, or any other article that has no fastening means for connecting to the wearer, but that is secured to the wearer by separate undergarment. The length of the flock may vary depending on needs, but preferably ranges between 0.2 and 20 mm, preferably 0.4 to 15 mm, more preferably 0.6 to 10 mm (referring to the visible portion of the flock). Viscose flock is particularly preferred. Other flocking materials include flock from natural and/or chemical raw materials or mixed forms.

In a further embodiment of the invention, the absorbent core includes one or more layers independently formed of terry cloth, fleece, molton or polyester, or any other material. The absorbent core takes up and retains the body fluid in its interior. Depending on needs and absorption capacity, it can have a single-layered or multilayered structure. For instance, the absorbent core may include one, two, three, four, five or more than five absorbent layers. Instead of or in addition to the number of absorbent layers, the absorption capacity can be modulated via the thickness of the absorbent core. If there are more than one absorbent layer, the absorbent layers can be the same or different. The absorbent core is preferably formed from one or more layers of woven, knitted, stitch-bonded, fleece or other fabrics. The fabric can be made from natural fibers, from synthetically or chemically produced threads and also includes mixed forms.

The various connections and joints described herein may be independently selected from the group consisting of seam joints, adhesive joints and welded joints. The advantage of a seam joint such as a (Zig-Zag) lockstitch or a 5-thread cover stitch or another sewing stitch type, is that the sanitary or incontinence article is repeatedly washable up to 90° C. On the other hand, adhesive joints and welded joints usually only withstand a wash temperature of at least 60° C. Generally, all seams referred to herein can be done by means of waterproof or water-repellent sewing thread.

Optionally, the sewing punctures are sealed by subsequently applying an adhesive coating material, preferably elastomer, in particular silicone, and/or threads that provide a sealing effect through heat or mechanical action. For instance, threads made of a thermoplastic elastomer may be heated so that the softened material fills the gaps. A swelling thread can also be used. The sewing punctures can also be sealed by applying, e.g. spraying, an adhering coating material as defined herein, such as silicone. In this case, conventional threads may be used. Using ultrasound, threads may be heated and softened, thereby filling any remaining gaps.

According to a further embodiment, the article of the invention further comprises front and rear covers folded over and sandwiching front and rear edges of the multilayer structure.

For example, the front and rear edges (from the perspective of the wearer) of the outer layer and the front and rear edges of the inner layer can be respectively trimmed with a folding ribbon or tape. The joint between the outer layer and the inner layer is preferably the same as the joint enclosing the absorbent core, but can also be a separate joint. It is further preferred that the joint is provided by means of any sewing stitch type. To prevent or reduce the risk that body fluid leaks through the front and rear covers, they are preferably formed from a waterproof or water-repellent material.

The front and rear may cover essentially cover entire respective front and rear edges of the multilayer structure. Further, side edges of front and rear covers may meet side edges of the side edge portions at respective corners of the multilayer structure. To reinforce the corners, portions of the front and rear covers can be sewn to adjacent portions of the side edge portions or processed in a different way. Preferably, the sewn portions include bartacks using, for instance, double backstitch or ZigZag stitching.

As will be appreciated by the skilled reader, the use of rear and front covers is particularly useful, but not limited to, a pantyliner or a pad. Therefore, in one embodiment, the reusable sanitary or incontinence article is a pantyliner or a pad.

A pantyliner is an absorbent piece of material used for feminine hygiene. It is worn in the gusset of a woman's panties for absorbance of daily vaginal discharge, light menstrual flow, tampon and menstrual cup backup, spotting, post-intercourse discharge, and urinary incontinence. Pantyliners are related to menstrual pads in their basic construction, but are usually thinner and often narrower than pads. As a result, they absorb less liquid than pads, making them ideal for light discharge and everyday cleanliness. They are generally unsuitable for the menstruation medium to heavy flow, which requires them to be changed more often.

A menstrual pad, or simply pad, is an absorbent item worn by women in their underwear when menstruating, bleeding after giving birth, recovering from gynecologic surgery, experiencing a miscarriage or abortion, or in any other situation where it is necessary to absorb a flow of blood from the vagina. A menstrual pad is worn externally. Pads are generally changed by being stripped off the pants and panties, taking out the old pad, sticking the new one on the inside of the panties and pulling them back on.

In another embodiment of the invention, the reusable sanitary or incontinence article is an underpants (for male or female) or a diaper.

An underpants is an underwear worn at the lower body of a male or female.

A diaper (also denoted as nappy) is a type of underwear that allows the wearer to urinate or defecate without using a toilet, by absorbing or containing waste products to prevent soiling of outer clothing or the external environment. Diapers are made of cloth or synthetic disposable materials. Cloth diapers are composed of layers of fabric, and can be washed and reused multiple times. Diapers are primarily worn by infants, toddlers who are not yet toilet trained, and by children who experience bedwetting. They are also used by adults under certain circumstances or with various conditions, such as incontinence.

Principally, the aforementioned features can be fully integrated into an underpants or a diaper. In particular, the use of front and rear covers does not exclude the article's use as a underpants or diaper. Rather, the multilayer structure including front and rear covers (with or without side edge portions) may be attached, e.g. sewn or adhered, to a pant-shaped or diaper-shaped base structure. Specifically, when the base structure has an inner surface facing the skin of the wearer and an outer surface facing away from the skin of the wearer, the multilayer structure can be attached in the crotch region to the inner surface of the base structure. However, in terms of a simple structure, a comfortable fit and an efficient use of material, it is preferred that the textile of the outer layer, in addition to a sealing portion, may include a base portion, which together with the sealing portion provide the shape and/or structure of a pant or a diaper. The base portion does not have to have, and preferably does not have, all layers of the multilayer structure.

The base portion may be coated with an elastomer. Further, the base portion may include flock sticking out of the elastomer coating, on its inner surface, i.e. the surface oriented towards and/or lying on the wearer's skin, and/or on its outer surface. The flock, when provided on the inner surface of the base portion, provides a comfortable feeling when worn. The flock, when provided on the outer surface of the base portion, may help to keep the article in place and/or may have an aesthetic function. As previously described, the flock can be applied to the inside or the outside or both the inside and the outside.

The elastomer used for the coating of the base portion is preferably the same as the elastomer used for the coating of the sealing portion, but may also be different. This may equally apply to the flock, provided that the sealing portion includes flock. This means, the flock in the sealing portion may be the same (e.g., of the same length and of the same material) as the flock in the base portion, or different. However, the sealing portion may also be free of flock, while the base portion may include flock, or both the sealing portion and the base portion may be free of flock, or both the sealing portion and the base portion may include flock. The following variants are mentioned specifically:
(i) The sealing portion includes flocked elastomer, the base portion is free of elastomer and flock.
(ii) The sealing portion and the base portion include flocked elastomer.
(iii) The sealing portion includes flocked elastomer, the base portion includes unflocked elastomer.
(iv) The sealing portion and the base portion include unflocked elastomer.

Unflocked elastomer coating may feel unpleasant if it is in direct contact with the skin. To avoid this, the elastomer coating may be covered by a textile layer. For instance, the textile layer may loosely overlie or may be adhered to the elastomer coating. The textile layer can be an additional layer that may be formed of the same material as the inner layer or of the same material as the textile of the outer layer. However, other materials are envisage herein.

Various edge finishing forms can be included in the underpants and diapers of the invention. For instance, the base portion can include flocked leg cut edges and/or flocked waist cut edges. As another example, the base portion can include folded leg cut edges and/or folded waist cut edges. For example, leg cut edge portions and/or waist cut edge portions of the base portion are folded over and connected to respective portions of the inner layer and/or of the base portion. Moreover, the base portion can include trimmed leg cut edges and/or trimmed waist cut edges. To this end, leg cut edges and/or waist cut edges may be trimmed by folding a ribbon or tape over and connecting it to respective edges. Leg cut edges at or adjacent the gusset area may be equally processed as in other leg cut edge regions. Leg cut edges can also be formed in such a way that an adhering coating material, preferably elastomer, in particular silicone, is introduced between the different layers of the multilayer structure to seal the layers against each other.

Underpants may further include joints at both sides (left and right from the perspective of a wearer). As described herein, the joint can be provided by stitching such as 4-thread cover stitching or overlock stitching, or other seams, by adhesive and/or welding.

The article, whether underpants, diaper, pantyliner or pad, may also include odour control agents and/or antimicrobial agents such as colloidal silver or activated carbon, optionally in a layer, for instance in one of the layers referred to herein and/or in a separate layer.

In another aspect of the invention, a method for producing a reusable sanitary or incontinence article having a multilayer structure as described herein, comprises the following steps:

Providing a first fabric layer defining an inner layer (as described herein) nearest to a wearer's skin for taking up body fluid from the wearer:
providing a second fabric layer defining an outer layer (as described herein);
providing one or more absorbent layers (as described herein) defining an absorbent core between the inner layer and the outer layer;
coating at least a portion of the second fabric layer (preferably contiguously) with an elastomer (as described herein), the portion defining a sealing portion (as described herein) providing a barrier for the body fluid;
superimposing the first fabric layer, the one or more absorbent layers and the second fabric layer such that the one or more absorbent layers is/are arranged between the first and second fabric layers, and the sealing portion extends at least over the gusset area (as described herein);
connecting the first layer, the second layer and optionally the one or more absorbent layers to each other along a perimeter of the gusset area by a fluid tight joint (as described herein);
cutting the first layer, the second layer and the one or more absorbent layers into shape (e.g., before or after the superimposing step, or before or after the connecting step) such that the second layer includes side edge portions (as described herein) extending from opposite sides outside the gusset area; and
folding the side edge portions over side edges of the first fabric layer and connecting them thereto.

The features and embodiment described in relation to the reusable sanitary or incontinence article are to be understood to describe corresponding features and embodiments of the method for producing said reusable sanitary or incontinence article. This means, for instance, when the outer layer has an outer surface facing away from the absorbent core and an inner surface facing towards the absorbent core, the method may further comprise coating the outer surface and/or the inner surface of the outer layer with the elastomer.

Moreover, the method may further include flocking the outer layer in the region of the elastomer coating. For the absorbent core, one or more layers independently formed of terry cloth, fleece, molton or polyester, or any other material can be used. The joint can be provided by seam joint, adhesive joint and/or welded joint or any other production step.

The method may further comprise folding front and rear covers over and sandwiching front and rear edges of the multilayer structure. Further, portions of the front and rear covers may be attached to adjacent portions of the side edge portions, e.g. by stitching.

The sanitary or incontinence article produced by the method of the invention is preferably selected from the group consisting of pantyliners, pads, underpants and diapers, and any other sanitary or incontinence article.

The outer layer may be cut into shape so as to include a base portion (in addition to the sealing portion), which together with the sealing portion provides the shape of a pant or a diaper. Also, the base portion may be coated with an adhereing coating material, preferably elastomer, in particular silicone, optionally the base portion may be flocked in a region of the coating. Furthermore, the method according to the invention may include flocking, folding or trimming leg cut edges and/or waist cut edges.

The concept underlying the present invention can be equally applied to an undergarment, in particular an underpants or a diaper or any other product, that is made liquid tight with an adhering coating material, preferably elastomer, in particular silicone, as described herein and that can be used with an external, e.g. separately provided, absorbent, e.g. a pantyliner, a pad or similar sanitary and incontinence articles. Thus, another aspect of the present invention concerns a reusable sanitary or incontinence undergarment comprising or consisting of a textile layer in the shape of the undergarment; a layer of an adhering coating material, preferably elastomer, in particular silicone, directly applied to the textile layer, the coating layer being contiguous, or designed in multiple parts, at least in the gusset area; and optionally a layer of flock embedded in and sticking out of the coating layer. The gusset area is configured to removably accept an absorbent for absorbing and retaining body fluids. The gusset area is free of permanent layers other than the textile layer, the coating layer and the flock layer. The term “permanent layer” denotes a sheet of material that is not detachable from the undergarment without compromising the integrity of the undergarment, in particular without causing damage. Accordingly, a removable absorbent is not understood as a permanent layer. Optionally, the undergarment include flocked, folded or trimmed leg cut edges and/or waist cut edges as described above.

Moreover, the textile layer, the coating and the flock of the undergarment of the invention may have the same characteristics as the corresponding components of the sanitary or incontinence article of the invention. Also, front, rear and side edges may be covered as described in relation to the sanitary or incontinence article of the invention. In addition, the undergarment can have a sealing portion and a base portion having the features described above.

A method for producing a reusable sanitary or incontinence undergarment is another aspect of the present invention. The method comprises providing a fabric layer; coating at least a portion of the fabric layer with an adhering coating material, the portion defining a sealing portion in the gusset area providing a barrier for the body fluid; cutting the fabric layer into shape; optionally flocking the elastomer coating to provide a flock layer; and optionally flocking, folding or trimming leg cut edges and/or waist cut edges. Further, the gusset area is free of permanent layers other than the fabric layer, textile layer or any other form of a layer, the coating and the flock layer. The coating is preferably contiguously at least in the gusset area.

As will be readily understood by the skilled reader, the further features of the undergarment of the invention require corresponding further steps and/or require the use of further and/or specific components. Such further steps and/or further components and/or specific components shall form corresponding embodiments of the production method of the invention. For instance, as described herein in relation to underpants and diapers, the undergarment can include various edge finishing forms. For instance, the textile can include flocked leg cut edges and/or flocked waist cut edges. As another example, the textile can include folded leg cut edges and/or folded waist cut edges. For example, leg cut edge portions and/or waist cut edge portions of the textile are folded over and connected to respective portions of the textile forming a double-layered textile structure. Moreover, the textile can include trimmed leg cut edges and/or trimmed waist cut edges. To this end, leg cut edges and/or waist cut edges may be trimmed by folding a ribbon or tape over and connecting it to respective edges. Leg cut edges at or adjacent the gusset area may be equally processed as in other leg cut edge regions. Further, pants may include joints at both sides (left and right from the perspective of a wearer). As described herein, the joint can be provided by stitching such as 4-thread cover stitching or overlock stitching, or any other sewing stitch type, or by adhesive or welding.

Most preferred embodiments of the invention are detailed in the following. As regards advantages, definitions and explanations, reference is made to the foregoing.

A reusable sanitary or incontinence article of the invention has a multilayer structure. The multilayer structure comprises an inner layer, an outer layer, an absorbent core and a fluid tight joint. The inner layer includes a gusset area nearest to a wearer's skin for taking up body fluid from the wearer and/or transports the body fluid away from the wearer. The outer layer includes a sealing portion for providing a barrier for the body fluid and side edge portions. The absorbent core is between the inner layer and the outer layer. The fluid tight joint connects the inner layer, the outer layer and optionally the absorbent core to each other along at least a part of a perimeter of the gusset area (i.e. at least along the side edges of the gusset area). The sealing portion is composed of a textile coated with an adhering coating material that forms a contiguous, impervious coating extending at least over the gusset area into the side edge portions. The side edge portions extend from opposite sides outside the gusset area, are folded over side edges of and connected to the inner layer forming a fluid tight joint between the inner layer and the outer layer along side edges of the gusset area. In this and the following embodiment, the side edge portions are part of (integral with) the outer layer, as opposed to a separate textile tape or the like.

In a preferred embodiment, the outer layer of the multilayer structure forms the outermost layer, or a layer adjacent to the outermost layer, of the reusable sanitary or incontinence article. In particular, the outer layer forms the outermost layer of the reusable sanitary or incontinence article in case of a women's underpants. This helps to keep the thickness of the underpants small. In case of a men underpants, the outer layer may form a layer adjacent the outermost layer of the reusable sanitary or incontinence article. While a small thickness is not of high importance for men underpants, it allows a more robust design. The reusable sanitary or incontinence article being a women's underpants or a men underpants is preferred.

Another preferred embodiment is where the outer layer has an outer surface facing away from the absorbent core and an inner surface facing towards the absorbent core, and where the outer surface and/or the inner surface of the outer layer is coated with the adhering coating material. The advantages achieved by this variation are explained above.

Preferred reusable sanitary or incontinence articles have on the outer layer flock embedded in the coating. The flock may be on the inner surface, or on the outer surface, or on both the inner and the outer surface, of the outer layer. The advantages achieved by this variation are explained above.

Preferably, the absorbent core includes one or more absorbent layers or pieces. Preferably the one or more layers or pieces are independently formed of, or include, terry cloth, fleece, molton, polyester or superabsorbent polymer. A particularly preferred feature of the invention is a piece of superabsorbent polymer that can be placed in, and removed from, a pocket formed in the article (as further explained herein). That is, the piece of superabsorbent polymer is removable. To ease introduction into the pocket, the piece may have an elongated shape, e.g., a cylindrical elongated or a rectangular elongated shape. Its dimension preferably ranges from 2 to 8 cm, preferably 3 to 7 cm, more preferably 4 to 6 cm, most preferably about 5 cm in width, and 10 to 35 cm, preferably 12 to 30 cm, more preferably 15 to 25 cm, most preferably about 20 cm in length. The thickness may vary in dependency on the desired absorbency and may generally range from 0.5 mm to 10 mm, preferably from 0.5 to 5.0 mm, more preferably from 1.0 to 2.0 mm.

A further preferred embodiment includes an open pocket formed in the multilayer structure for replacing the absorbent core, or one or more of the absorbent layers or pieces thereof. The open pocket may be accessible from outside (side facing away from wearer) or the inside (side facing the wearer) of the reusable sanitary or incontinence article. It is to be understood that this variation cannot only be implemented into underpants but also other articles such as pads as illustrated in FIGS. 9 and 10.

A particularly preferred structure of an absorbent core includes a piece of superabsorbent polymer (as defined above) encased by at least two textile based absorbent layers. Preferably, the piece of superabsorbent polymer is replaceable and the at least two textile based absorbent layers are fixedly attached to the multilayer structure and form an open pocket. The at least two textile based absorbent layers are preferably configured to readily absorb and transport body fluids only, or at least mainly, in the direction of the superabsorbent polymer piece. Thereby, the superabsorbent polymer piece is given sufficient time to absorb the body fluid and yet provide a dry feeling.

In preferred reusable sanitary or incontinence articles, the joint is selected from the group consisting of seam joints, adhesive joints and welded joints, and combinations thereof. It is further preferred that the joint is formed by a combination of a seam joint and an adhesive joint, or a combination of a seam joint and a welded joint. The advantage of this structure become apparent from the following, further specified embodiment.

According to a specific embodiment, the outer layer has an outer surface facing away from the absorbent core and an inner surface facing towards the absorbent core. The inner layer has an outer surface facing away from the absorbent core and an inner surface facing towards the absorbent core. The joint includes a hardened melt film on the inner surface of the outer layer and/or a hardened melt film on the inner surface of the inner layer. The hardened melt film at least partially seals thread piercing holes of the seam joint. By sealing from the inside of the multilayer structure, the hardened melt film does not create an unpleasant feeling and does not interfere with ironing. At the same time, the seam joint helps the joint to securely withstand washing and drying at around 90° C.

The melt film preferably comprises or consists of a thermoplastic polymer, in particular thermoplastic polyurethane.

According to a further preferred embodiment, the outer layer includes a rear cover portion sealingly connecting the multi-layer structure, wherein the contiguous, impervious coating extends into the rear cover portion, wherein the rear cover portion is folded over a rear edge of and connected to the inner layer forming a fluid tight joint between the inner layer and the outer layer along the rear edge of the gusset area. The rear cover portion is understood to be part of the outer layer. The embodiment does not feature a front cover portion, but allows at this position access to an open pocket formed in the multilayer structure for removably introducing an absorbent core.

In alternative embodiment, the outer layer includes rear and front cover portions sealingly connecting the multilayer structure, wherein the contiguous, impervious coating extends into the rear and front cover portions, wherein the rear and front cover portions are folded over respective rear and front edges of and connected to the inner layer forming a fluid tight joint between the inner layer and the outer layer along the rear and front edges of the gusset area. According to this alternative embodiment, a circumferential joint is formed encircling the gusset area, and the absorbent core is not replaceable.

In further embodiments, a sealing tape extends widthwise across the side edge portions and the rear cover portion and, if present, the front cover portion.

The reusable sanitary or incontinence article of the present invention can be selected from the group consisting of pantyliners, pads, underpants and diapers as described elsewhere herein. For example, a pad in the form of the multilayer structure as described herein may be used for being placed in conventional underpants. Alternatively, a pad in the form of the multilayer structure as described herein may be provided or used as a pre-assembly for attachment, e.g. suturing or gluing, to underpants. The attachment may be spot or full surface. Further, a pad may take a variety of shapes and sizes. For example, the pad may cover the entire width at the back part seam.

A method for producing a reusable sanitary or incontinence article as described above comprises the following steps: A first textile layer defining an inner layer (as described herein) nearest to a wearer's skin including a gusset area for taking up body fluid from the wearer and/or for transporting the body fluid away from the wearer is provided. A second textile layer defining an outer layer (as described herein) is provided. One or more absorbent layers and/or pieces defining an absorbent core (as described herein) between the inner layer and the outer layer is/are provided. At least a portion of the second textile layer is coated with an adhering coating material. The portion defines a sealing portion (as described herein) providing a barrier for the body fluid. The first textile layer, the one or more absorbent layers and/or pieces and the second textile layer are superimposed such that the one or more absorbent layers and/or pieces is/are arranged between the first and second textile layers, and the sealing portion extends at least over the gusset area into side edge portions (as described herein) of the second layer. The first layer, the second layer and optionally the one or more absorbent layers and/or pieces are connected to each other along at least a part of a perimeter of the gusset area by a fluid tight joint (as described herein). Optionally, the first layer, the second layer and the one or more absorbent layers and/or pieces are cut into shape. The side edge portions of the second layer are sealingly connected to the multilayer structure. Further, the side edge portions extend from opposite sides outside the gusset area, are folded over side edges of the first textile layer and connected to the inner layer forming a fluid tight joint between the inner layer and the outer layer along side edges of the gusset area.

Another reusable sanitary or incontinence undergarment of the present invention comprises or consists of the following: A first textile layer is in the shape of the undergarment and forms the outermost layer of the undergarment. A second textile layer next to the first textile layer is optional (mainly depending whether the underpants are for women or men). A third layer forms an innermost layer including a gusset area nearest to a wearer's skin. A layer of an adhering coating material directly applied to the first textile layer and/or, if present, to the second textile layer, forms a contiguous, impervious coating extending at least over the gusset area. A layer of flock is embedded in and sticking out of the coating layer. Further, the gusset area is configured to removably accept an absorbent core for absorbing and retaining body fluids.

A reusable sanitary or incontinence undergarment as described above can be produced by a method comprising the steps of providing a first textile layer; optionally, providing a second textile layer; providing a third textile layer; coating at least a portion of the first textile layer and/or, if present, at least a portion of the second textile layer, with an adhering coating material, the portion defining a sealing portion extending at least over the gusset area for providing a barrier to the body fluid; optionally, cutting the first and third textile layers and, if present, the second textile layer into shape; flocking the coating to provide a flock layer; and connecting the first textile layer with the third textile layer along a part of a perimeter of the gusset area so that an open pocket is formed for accepting and/or replacing and absorbent core, or one or more absorbent layers or pieces thereof.

Finally, it is noted that each feature described in relation to a specific aspect or embodiment is meant to be combinable with other aspects or embodiments, unless the combination is technically meaningless or impossible.

### BRIEF DESCRIPTION OF THE DRAWINGS

The inventive concept, non-limiting embodiments, and further advantages of the inventive concept will now be described with reference to the drawings in which:

FIG. 1 illustrates exemplary multilayer structures of sanitary or incontinence articles.

FIG. 2a-e illustrate an exemplary method for producing a multilayer structure.

FIGS. 3-4 illustrate finishing forms of open cut parts of underpants configured to be used with exchangeable absorbents.

FIG. 5 shows exemplary exchangeable absorbents.

FIGS. 6 and 7 show underpants providing access to a removable absorbent core.

FIGS. 8 and 9 show pads providing access to a removable absorbent core.

FIGS. 10, 11 and 12 are examples of men underpants having a coated and flocked layer next to an outermost layer and a permanent and/or replaceable absorbent.

FIG. 13 illustrates another example of articles of the invention in the form of a pad.

FIG. 14 shows an exemplary method of production and/or assembly of men underpants according to an embodiment of the invention.

FIG. 15 shows variations of the opening positions of open pocket of exemplary men underpants.

FIG. 16 illustrates the production and/or assembly of women underpants according to an embodiment of the invention

FIG. 17 illustrates the folding of the side edges of the underpants base extending outside of the gusset area over the side edges of the inner layer in detail.

FIG. 18 is a cross sectional view of the crotch region of the women underpants produced according to the process shown in FIG. 16.

FIG. 19 is a cross sectional view of the crotch region of the women underpants of FIG. 18 and illustrates the sealing from the inside.

FIG. 20 illustrates opening (slide-in) positions for introducing and removing a removable absorbent core.

### DETAILED DESCRIPTION OF THE DRAWINGS

The description of the invention to be provided herein is given purely by way of example and is not to be taken in any way as limiting the scope or extent of the invention. The invention will be described with reference to an example range of undergarments, as underpants, shorts, briefs or panties, or slips.

In the following description, the present inventive concept is described with reference to reusable, washable and dryable sanitary and incontinence articles.

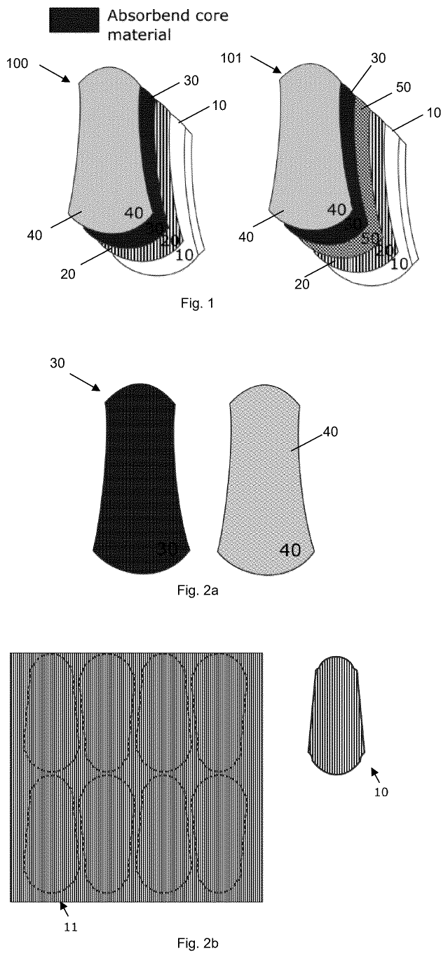

FIG. 1 illustrates exemplary multilayer structures 100, 101 of a sanitary or incontinence article. Structure 100 is conformable to a human body and consists of an outer layer 10, an adhesive coating layer 20, an absorbent core 30 and an inner layer 40. When worn, inner layer 40 is next to the skin of the crotch area. Absorbent core 30 is arranged between inner layer 40 and coating layer 20. Coating layer 20 is between outer layer 10 and absorbent core 30, for instance coating layer 20 adheres over its entire surface to the inner surface of outer layer 10. However, coating layer 20 can also adhere over its entire surface to the outer surface of outer layer 10.

Structure 101 is conformable to a human body and consists, in this order, of an outer layer 10, a coating layer 20, a flock layer 50, an absorbent core 30 and an inner layer 40. Coating layer 20 adheres over its entire surface to the inner surface of outer layer 10. Similarly, flock layer 50 adheres over its entire surface to the inner surface of coating layer 20. However, coating layer 20 can also adhere over its entire surface to the outer surface of outer layer 10, whereas flock layer 50 may adhere over its entire surface to the outer surface of coating layer 20.

While structures 100, 101 are shown and described to consist of the described layers, they may principally include further layers. Further, structures 100, 101 take the shape of a sanitary or incontinence pad. However, they can also be part of another sanitary or incontinence article such as an underwear. Moreover, while absorbent core 30 is shown as one layer, it may be composed from more than one layers such as 2, 3, 4, 5 or more layers.

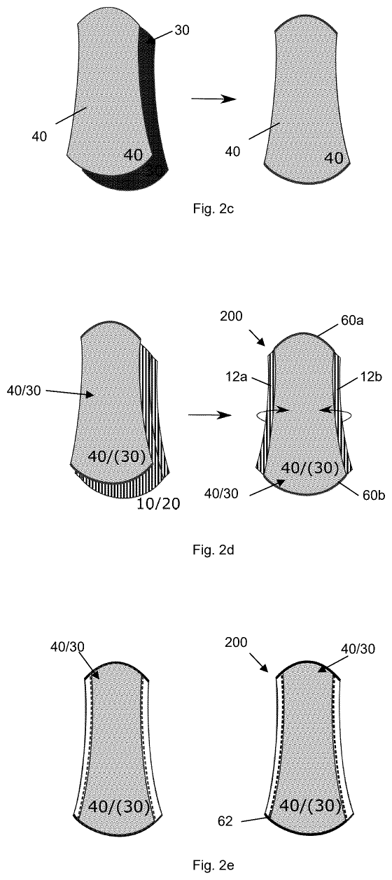

FIG. 2a-e illustrate an exemplary method for producing multilayer structure 200, which can be identical to one of multilayer structures 100, 101. As seen in FIG. 2a, absorbent core 30 and inner layer 40 are provided in fine cut, or rough cut or in another form, in this example in the shape of a pad to be produced, whereas a material 11 from which one or more outer layers 10 can be cut out, is provided in rough cut. Material 11 is coated with adhering coating material forming coating layer(s) 20 on outer layer(s) 10. Optionally, the coating can be flocked. After drying, e.g. in the drying oven or in the hot press, material 11 is cut into shape leaving side edge portions 12a, 12b extending over respective side edges of the absorbent core 30 and inner layer 40. FIG. 2b shows that the layers are stacked on top of each other in the desired order. Side edge portions 12a, 12b are then folded over respective side edges of the absorbent core 30 and inner layer 40, and attached to inner layer 40 (cf. FIG. 2c). Attachment can be achieved by hot pressing glue, adhesive coating material, preferably elastomer, in particular silicone, by sewing or welding or any other form, between folded side edge portions 12a, 12b and inner layer 40, and/or by stitching folded side edge portions 12a, 12b onto inner layer 40. If desired, front and rear edges of the multilayer structure can be trimmed with respective front and rear covers 60a, 60b (cf. FIG. 2d). Further, corner seams 62 may extend across corners of the multilayer structure such that portions of rear covers 60a, 60b are attached to adjacent portions of side edge portions 12a, 12b (cf. FIG. 2e).

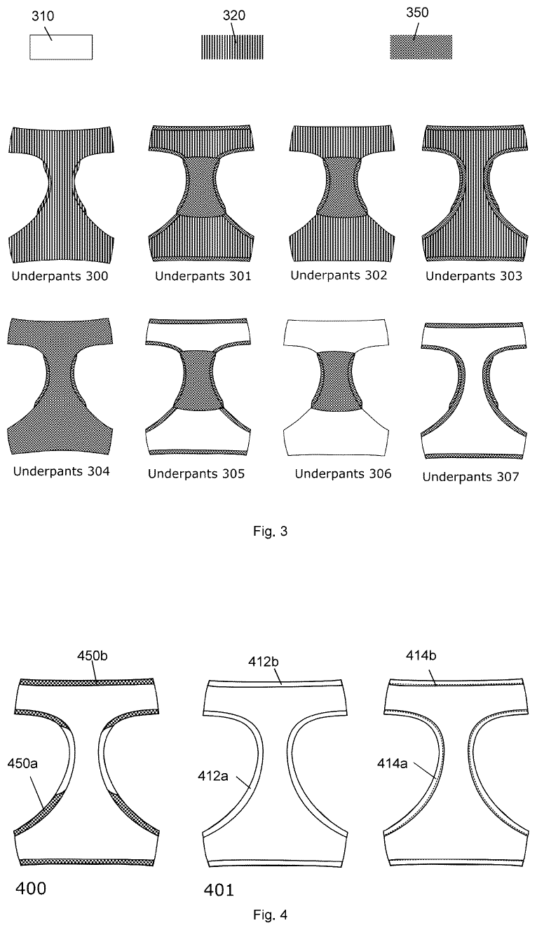

FIGS. 3-4 show open cut parts of underpants configured to be used with an exchangeable absorbent. FIG. 3 shows examples of different flocking patterns. Generally, the underpants are made up of a conformable outer layer 310 in the form of an underpants and an elastomer layer 320 on the inside of the outer layer at least in the crotch region/gusset area. In these examples, elastomer layer 320 extends over the whole inner surface of the outer layer. In underpants 300, elastomer layer 320 is free of flock. Underpants 301 is flocked only in the gusset area with a flock layer 350. In underpants 302, flock layer 350 extends over the whole surface of the elastomer layer. Further variations are shown in underpants 303, 304, 305, 306, 307. In particular, in underpants 300 the coated outer layer is free of flock over the whole inner surface of the outer layer. In underpants 301, the coated outer layer contains flock only in the gusset and leg opening/waistband areas. In underpants 302, the coated outer layer contains flock only in the gusset area. In underpants 303, the coated outer layer contains flock only in the leg opening/waistband areas. In underpants 304, the coated outer layer contains flock over the whole inner surface of the outer layer. In underpants 305, the coated outer layer contains coating and flock only in the gusset and leg opening/waistband areas. In underpants 306, the coated outer layer contains coating and flock only in the gusset area. In underpants 307, the coated outer layer contains coating and flock only in the leg opening/waistband areas.

FIG. 4 shows different possibilities to finish leg and waist cuts. The finishing forms can be combined with any multilayer structure and flocking pattern described herein. Underpants 400 illustrates flocked leg cut edges 450a and flocked waist cut edges 450b. Underpants 401 includes folded leg cut edge portions 412a and folded waist cut edge portions 412b. The folded cut edge portions 412a, 412b can be attached to the outer layer, flock layer and/or coating layer by glue or stitching or ultrasound or any other bonding method. The leg cut out portions in the gusset area can be processed similarly.

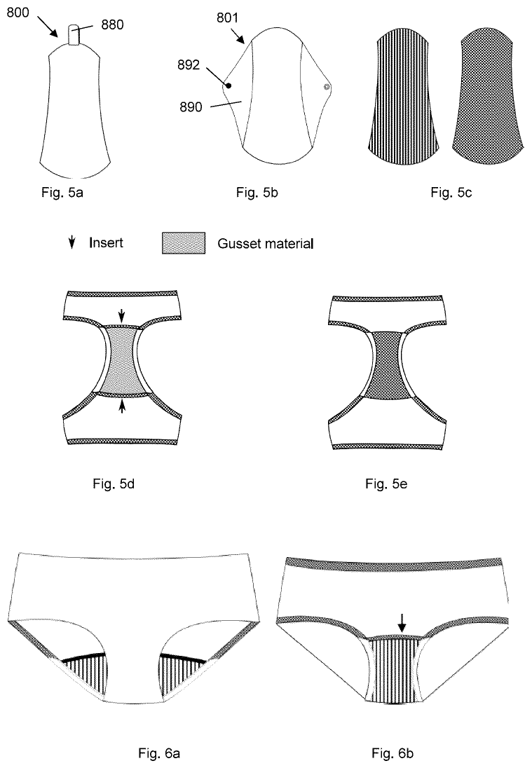

Exemplary exchangeable absorbents are shown in FIG. 5. The underpants and/or the absorbent may include means for reversible connecting both together. For instance, the underpants may include a pocket into which the absorbent can be inserted (cf. FIG. 5d). To ease gripping and removing of the absorbent from the pocket, absorbent 800 is provided with a strap or tab 880, as shown in FIG. 5a. As shown in FIG. 5b, absorbent 801 can also include side wings 890, which can be connected with the underpants, e.g. by means of buttons 892 or other connection means such as Velcro fastener and the like.

The absorbents may have the same multilayer structure as the multilayer structure of the herein described articles. However, when the underpants is sealed by an elastomer layer directly adhered to the underpants/outer layer, the exchangeable absorbent may principally omit the elastomer layer. However, the elastomer coating and/or the flock layer, when applied onto the outermost surface, as illustrated in FIG. 5c, may not only safeguard dryness but may also prevent slipping of the exchangeable absorbent within the underpants, in particular, when the absorbent and loosely placed in the underpants, as shown in FIG. 5e.

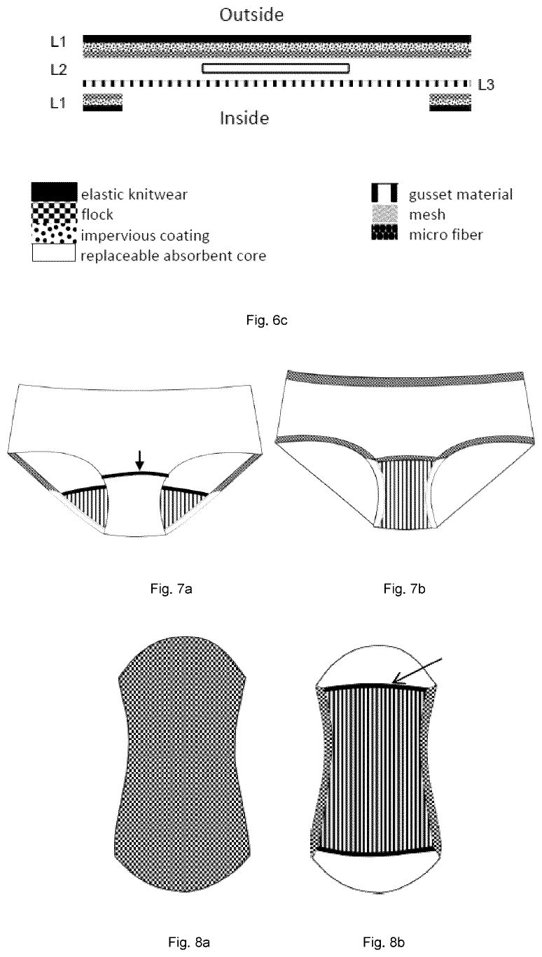

FIG. 6 shows underpants from the outside (FIG. 6a) and the inside (FIG. 6b), and in cross section (FIG. 6c). In this example, the inside provides access (indicated by the arrow) to an open pocket for receiving an absorbent core or a layer or piece thereof. For instance, a layer of microfiber material or a piece of superabsorbent material can serve as replaceable absorbent. The layers of the exemplary underpants in cross section are identified in FIG. 6c. Specifically, the outer layer (L1) comprises elastic knitwear (polyester/elastane) provided with an impervious and flocked coating. The absorbent core (L2), in this example, is replaceable, but may in other examples be permanent. The inner layer (L3) denotes the gusset material. FIG. 6c further illustrates with the lower L1-layer the folding of the outer layer over side portions of the gusset material.

FIG. 7 shows another underpants that provides access to the open pocket from the outside of the underpants as indicated by the arrow in FIG. 7a. Otherwise, the multilayer structure is the same as shown in FIG. 6c.

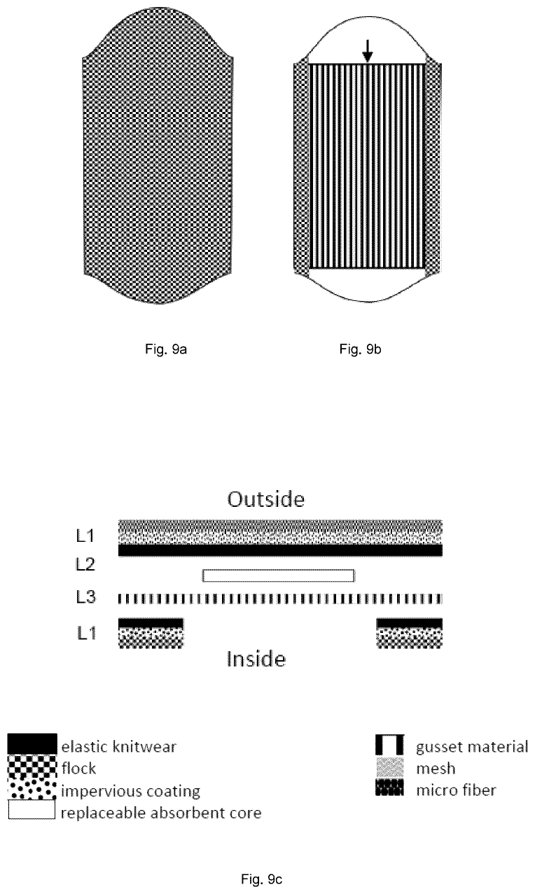

FIGS. 8 and 9 show pads providing access to an open pocket as indicated by the arrows in FIGS. 8b and 9b. The open pocket may be configured to receive an absorbent. The layers of the exemplary pad in cross section are identified in FIG. 9c. The structure is similar to that shown in FIG. 6c, except that the flock forms the outermost layer and the elastic knitwear (polyester/elastane) is closer to the user than the flock (L1). The absorbent core (L2), in this example, is replaceable, but may in other examples be permanent. In addition, a replaceable absorbent can be combined with a non-replaceable absorbent. The inner layer (L3) denotes the gusset material. FIG. 9c further illustrates that the outer layer (L1) is folded over side portions of the gusset material (L3).

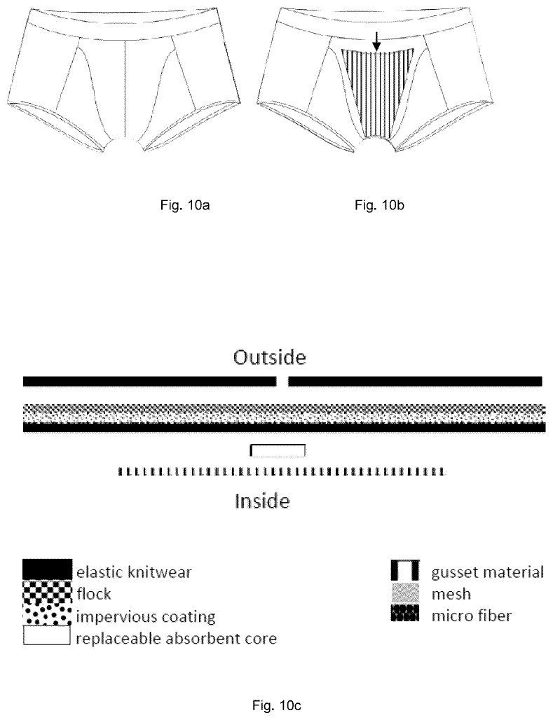

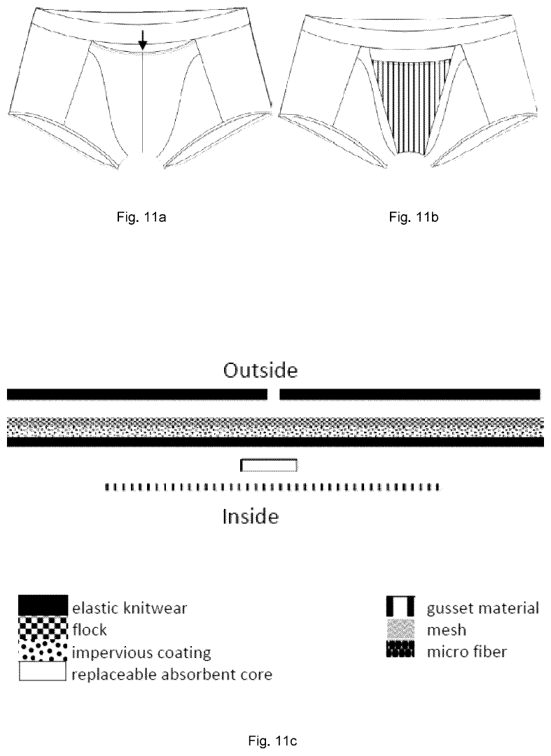

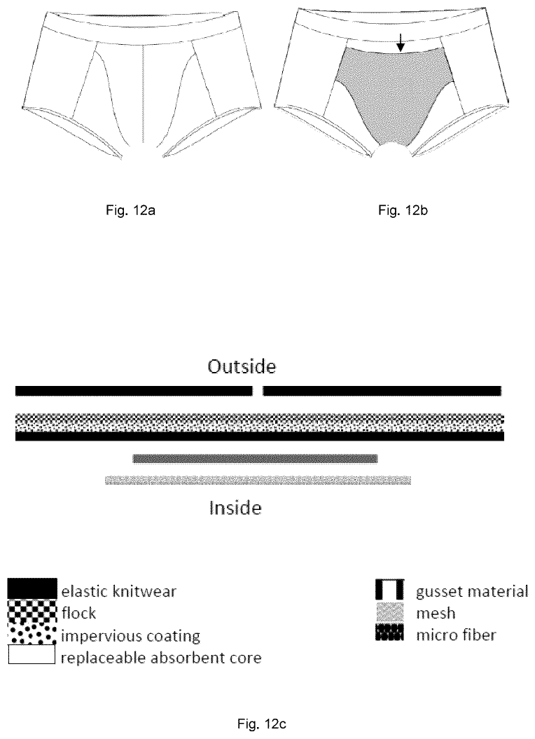

FIGS. 10, 11 and 12 are examples of men underpants having elastic knitwear (cotton/elastane) as outermost layer, a coated and flocked layer of elastic knitwear (polyester/elastane) next to the outermost layer, a permanent or replaceable absorbent and a mesh (FIG. 12) or gusset material (FIG. 10, 11) as innermost layer. In the example of FIGS. 10 and 12, an open pocket is accessible from the inside of the underpants (FIGS. 10b, 12b). In the example of FIG. 11, an open pocket is accessible from the outside of the underpants (FIG. 11b). The corresponding multilayer structures are shown in FIGS. 10c, 11c and 12c.

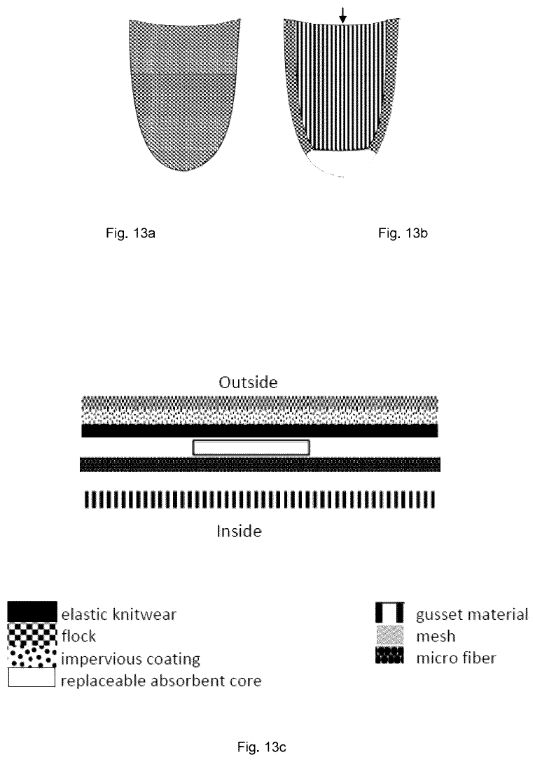

FIG. 13 illustrates another example of a pad, similar to those shown in FIGS. 8 and 9.

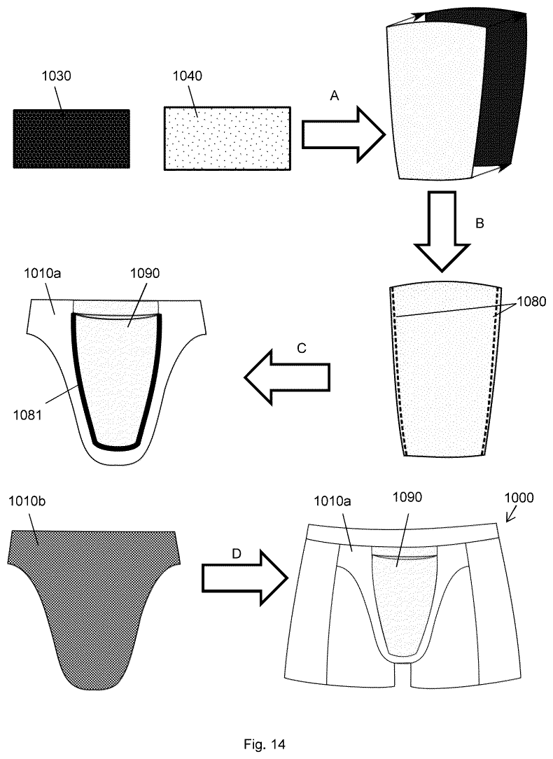

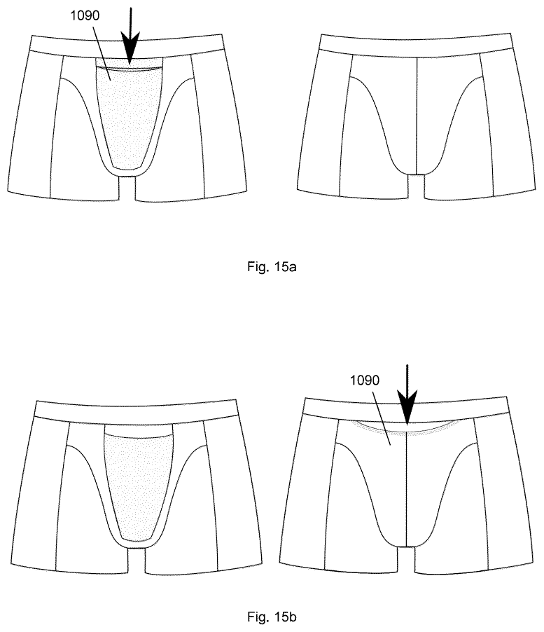

FIG. 14 illustrates an exemplary method of production and/or assembly of men underpants according to an embodiment of the invention. An absorbent core material forming the absorbent core 1030 (one or more layers) and a gusset material forming the inner layer 1040 are provided. The layers are piled and connected onto each other along their side edges using seam/stitching 1080 (Steps A and B). The structure is then attached, e.g. sutured, in the crotch region onto the inside of the outer layer 1010a along the side edges and the lower edge (along below sealing/glue tape 1081). An open pocket 1090 is formed between the outer layer 1010a and inner layer 1040 for introducing a replaceable absorbent core. The seam can be covered by a sealing tape 1081 (Step C). The backside of the outer layer 1010b is made impervious with an elastomer layer that can be flocked or unflocked. In this example, the elastomer layer is flocked on the left fabric side. The layer structure is then attached in the crotch region of men underpants 1000 (Step D). In the final underpants 1000, reference sign 1010a indicates the leakage protection area, i.e. the area which is impervious thus prevents body liquid from dripping through the underpants 1000.

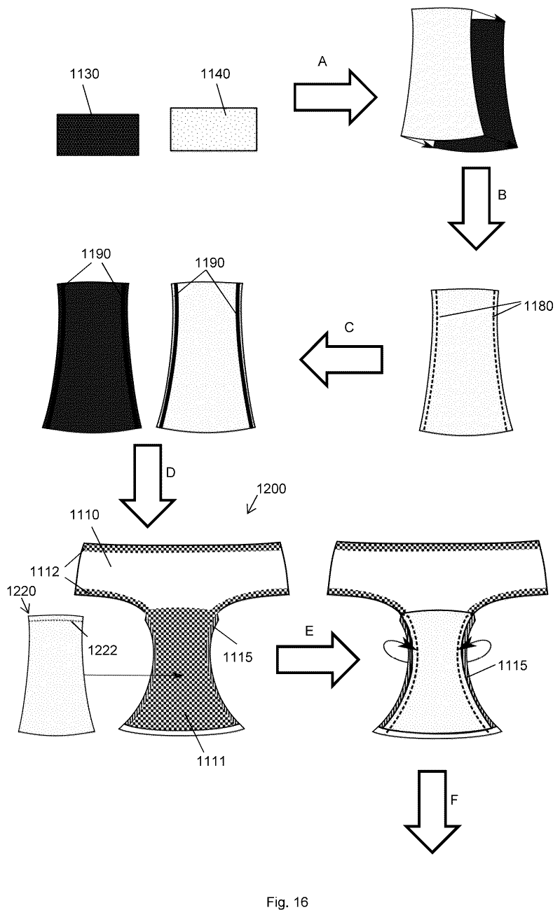

FIG. 15 shows variations of the opening positions of open pocket 1090, 1090a. In FIG. 15a, which shows an inside view (onto the left fabric side) on the left and an outside view (onto the right fabric side) on the right, the opening of the open pocket 1090 is formed on the inside (left fabric side) of the underpants similar to FIG. 14. In FIG. 15b, the opening of the open pocket 1090a is formed on the outside (right fabric side) of the underpants.

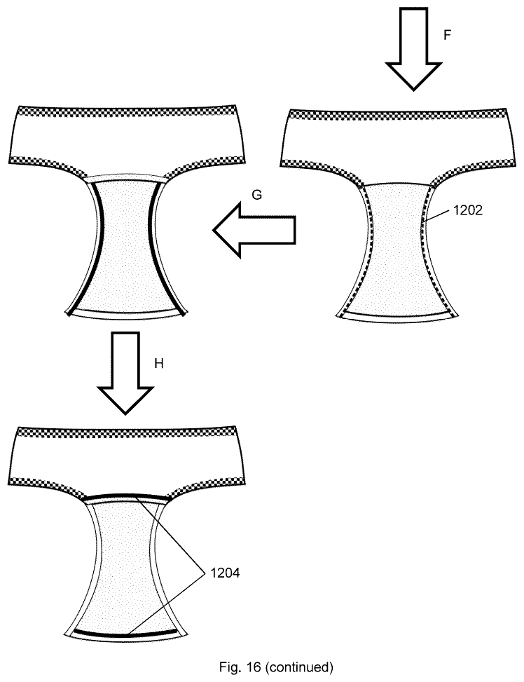

FIG. 16 illustrates the production and/or assembly of women underpants. An absorbent core material forming the absorbent core 1130 (one or more layers) and a gusset material forming the inner layer 1140 are provided. The layers are piled and connected onto each other along their side edges using seam/stitching 1180 (Steps A and B). On both sides of the structure, a sealing tape 1190 is attached to the side edge regions (Step C). Optionally, the flocked side can be covered with a mesh layer.

A women underpants base 1200 is provided. The base 1200 has the shape of women underpants and is constituted by an outer layer 1110 that includes coated and, optionally, flocked portions. Region 1111 denotes the sealing portion that is coated with an elastomer to render the portion impervious. Side edges 1115 of the sealing portion 1111 extending outside the gusset area are free of flock, whereas the remainder of the sealing portion 1111 is flocked. Region 1112 denotes leg cut out and waist edges that are coated and flocked.

The upper edge of the layered structure 1220 comprising the inner layer 1140 and the absorbent core 1130 is folded and sewn along stitching line 1222. Structure 1220 is then attached in the crotch region of the base 1200 (Step D).

The structure 1220 is then attached, e.g. sutured, in the crotch region onto the inside of the base 1200 along the stitching line near the side edges of structure 1220. The side edges 1115 of base 1200 extending outside of the gusset area are folded over the side edges of the structure 1220 and sutured thereto (Step E). The stitching 1202 in the side edge region is then sealed from the inside by heat-melting the sealing tape added in Step C (Steps F, G). Finally, a sealing tape is attached on the upper and lower edges 1204 of the gusset region.

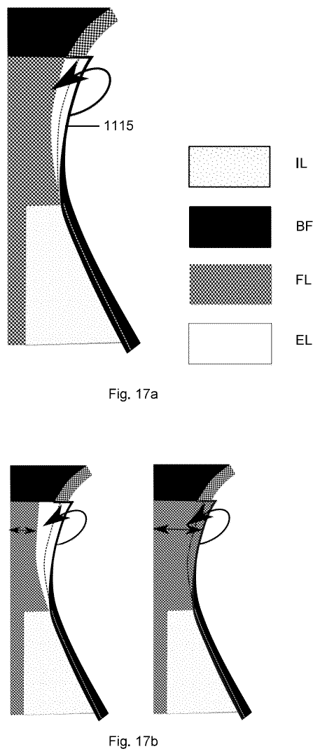

FIG. 17 shows step E, i.e. the folding of the side edges 1115 of the underpant base extending outside of the gusset area over the side edges of the inner layer (IL), in more detail. The layers are shown in different patterns. IL=inner layer (gusset), BF=body fabric, FL=flock layer/coating, EL=elastomer layer/coating free of flock. It can be seen that, in the example of FIG. 17a, the side edges 1115 folded over the inner layer (IL) are elastomer coated and free of flock. FIG. 17b illustrate that the width of the flock free area is variable. As shown on the left side of FIG. 17b, the flock free region is rather wide, whereas on the right side of FIG. 17b, the side edges are completely flocked, i.e. there is no flock free area in the side edge region.

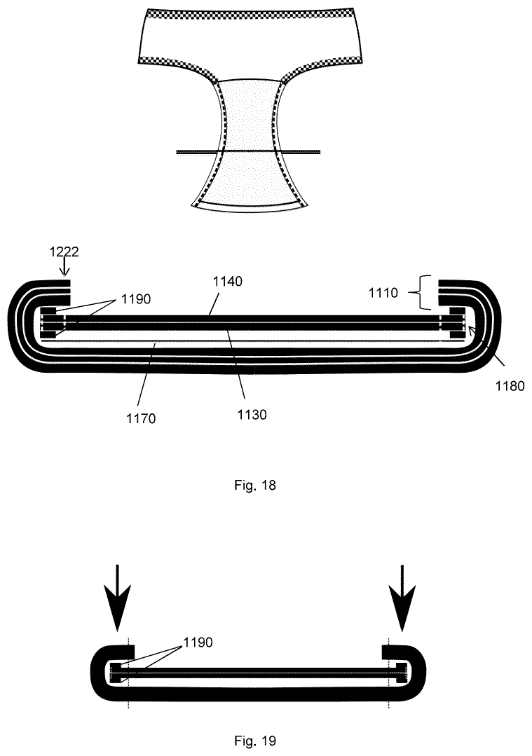

FIG. 18 is a cross sectional view of the crotch region of the women underpants produced according to the process shown in FIG. 16. It is shown absorbent core 1130, inner layer 1140, seam/stitching line 1180, sealing tape 1190, coated and flocked outer layer 1110, seam/stitching line 1222, optional mesh layer 1170 (not shown in FIG. 16).

FIG. 19 is a cross sectional view of the crotch region of the women underpants of FIG. 18 and illustrates the sealing from the inside by heat-melting the sealing tape 1190 included in the layer structure through application of heat and pressure in the direction indicated by the arrows.

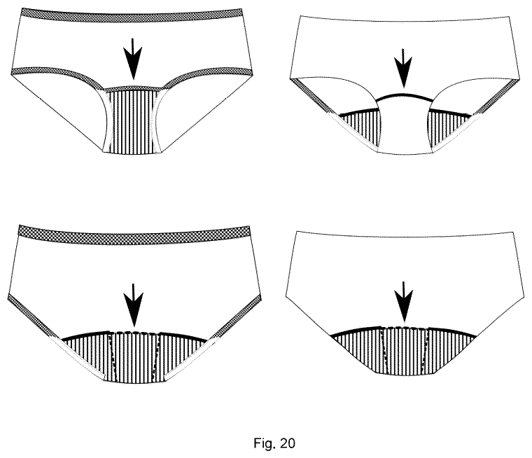

FIG. 20 illustrates opening (slide-in) positions for introducing and removing a removable absorbent core. The arrows indicate the positions of the openings and the directions of introducing the absorbent core. The illustrated positions are on the inside of the front side (upper left), on the outside of the front side (upper right), on the inside of the back side (lower left) and on the outside of the back side (lower right).

1. A reusable sanitary or incontinence article having a multilayer structure comprising:
an inner layer including a gusset area nearest to a wearer's skin for taking up body fluid from the wearer and/or transporting the body fluid away from the wearer;
an outer layer including a sealing portion for providing a barrier for the body fluid and side edge portions;
an absorbent core between the inner layer and the outer layer; and
a fluid tight joint connecting the inner layer, the outer layer and optionally the absorbent core to each other along at least a part of a perimeter of the gusset area;
wherein the sealing portion is composed of a textile coated with an adhering coating material that forms a contiguous, impervious coating extending at least over the gusset area into the side edge portions, and
wherein the side edge portions extend from opposite sides outside the gusset area, are folded over side edges of and connected to the inner layer forming a fluid tight joint between the inner layer and the outer layer along side edges of the gusset area.

2. The reusable sanitary or incontinence article of claim 1, wherein the outer layer of the multilayer structure forms the outermost layer, or a layer adjacent to the outermost layer, of the reusable sanitary or incontinence article.

3. The reusable sanitary or incontinence article of claim 1, wherein the outer layer has an outer surface facing away from the absorbent core and an inner surface facing towards the absorbent core, and wherein the outer surface and/or the inner surface of the outer layer is coated with the adhering coating material.

4. The reusable sanitary or incontinence article of claim 1, wherein the outer layer includes flock embedded in the coating, wherein the flock is on the inner surface, or on the outer surface, or on both the inner and the outer surface, of the outer layer.

5. The reusable sanitary or incontinence article of claim 1, wherein the absorbent core includes one or more absorbent layers or pieces.

6. The reusable sanitary or incontinence article of claim 5, wherein the multilayer structure forms an open pocket for replacing the absorbent core, or one or more of the absorbent layers or pieces thereof.

7. The reusable sanitary or incontinence article of claim 1, wherein the absorbent core includes a piece of superabsorbent polymer encased by at least two textile based absorbent layers.

8. The reusable sanitary or incontinence article of claim 1, wherein the joint is selected from the group consisting of seam joints, adhesive joints, welded joints, and combinations thereof.

9. The reusable sanitary or incontinence article of claim 7, wherein the outer layer has an outer surface facing away from the absorbent core and an inner surface facing towards the absorbent core, wherein the inner layer has an outer surface facing away from the absorbent core and an inner surface facing towards the absorbent core, wherein the joint includes a hardened melt film on the inner surface of the outer layer and/or a hardened melt film on the inner surface of the inner layer, wherein the hardened melt film at least partially seals thread piercing holes of the seam joint.

10. The reusable sanitary or incontinence article of claim 1,
wherein the outer layer includes a rear cover portion sealingly connecting the multilayer structure, wherein the contiguous, impervious coating extends into the rear cover portion, wherein the rear cover portion is folded over a rear edge of and connected to the inner layer forming a fluid tight joint between the inner layer and the outer layer along the rear edge of the gusset area; or
wherein the outer layer includes rear and front cover portions sealingly connecting the multilayer structure, wherein the contiguous, impervious coating extends into the rear and front cover portions, wherein the rear and front cover portions are folded over respective rear and front edges of and connected to the inner layer forming a fluid tight joint between the inner layer and the outer layer along the rear and front edges of the gusset area.

11. The reusable sanitary or incontinence article of claim 9, wherein a sealing tape extends widthwise across the side edge portions and the rear cover portion and optionally the front cover portion.

12. The reusable sanitary or incontinence article of claim 1, wherein the sanitary or incontinence article is selected from the group consisting of pantyliners, pads, underpants, and diapers.

13. A method for producing a reusable sanitary or incontinence article of claim 1, comprising:
providing a first textile layer defining an inner layer nearest to a wearer's skin including a gusset area for taking up body fluid from the wearer and/or for transporting the body fluid away from the wearer;
providing a second textile layer defining an outer layer;
providing one or more absorbent layers and/or pieces defining an absorbent core between the inner layer and the outer layer;
coating at least a portion of the second textile layer with an adhering coating material, the portion defining a sealing portion providing a barrier for the body fluid;
superimposing the first textile layer, the one or more absorbent layers and/or pieces and the second textile layer such that the one or more absorbent layers and/or pieces is/are arranged between the first and second textile layers, and the sealing portion extends at least over the gusset area into side edge portions of the second layer;
connecting the first layer, the second layer and optionally the one or more absorbent layers and/or pieces to each other along at least a part of a perimeter of the gusset area by a fluid tight joint;
optionally, cutting the first layer, the second layer, and the one or more absorbent layers and/or pieces into shape;
sealingly connecting the side edge portions of the second layer to the multilayer structure,
wherein the side edge portions extend from opposite sides outside the gusset area, are folded over side edges of the first textile layer, and connected to the inner layer forming a fluid tight joint between the inner layer and the outer layer along side edges of the gusset area.

14. A reusable sanitary or incontinence undergarment comprising:
a first textile layer in the shape of the undergarment forming the outermost layer of the undergarment;
optionally, a second textile layer next to the first textile layer;
a third layer forming an innermost layer including a gusset area nearest to a wearer's skin;
a layer of an adhering coating material directly applied to the first textile layer and/or, if present, to the second textile layer, the coating layer forming a contiguous, impervious coating extending at least over the gusset area; and
a layer of flock embedded in and sticking out of the coating layer;
wherein the gusset area is configured to removably accept an absorbent for absorbing and retaining body fluids.

15. A method for producing a reusable sanitary or incontinence undergarment of claim 14, comprising:
providing a first textile layer;
optionally, providing a second textile layer;
providing a third textile layer;
coating at least a portion of the first textile layer and/or, if present, at least a portion of the second textile layer, with an adhering coating material, the portion defining a sealing portion extending at least over the gusset area for providing a barrier to the body fluid;
optionally, cutting the first and third textile layers and, if present, the second textile layer into shape;
flocking the coating to provide a flock layer; and
connecting the first textile layer with the third textile layer along a part of a perimeter of the gusset area so that an open pocket is formed for accepting and/or replacing and absorbent core, or one or more absorbent layers or pieces thereof.

16. The reusable sanitary or incontinence article of claim 5, wherein the one or more absorbent layers or pieces are independently formed from or include terry cloth, fleece, molton, polyester, or a superabsorbent polymer.

17. The reusable sanitary or incontinence article of claim 7, wherein the piece of superabsorbent polymer is replaceable and the at least two textile based absorbent layers are fixedly attached to the multilayer structure and form an open pocket.

18. The reusable sanitary or incontinence article of claim 8, wherein the joint is formed by a combination of a seam joint and an adhesive joint, or a combination of a seam joint and a welded joint.

19. The reusable sanitary or incontinence article of claim 9, wherein the melt film comprises a thermoplastic polymer.

20. The reusable sanitary or incontinence article of claim 19, wherein the thermoplastic polymer is thermoplastic polyurethane.
